(19) United States
(12) Patent Application Publication
(10) Pub. No.: US 2023/0332236 A1
(43) Pub. Date: Oct. 19, 2023

(54) METHOD FOR DIAGNOSING STROKE UTILIZING GENE EXPRESSION SIGNATURES

(71) Applicant: Ischemia Care LLC, Oxford, OH (US)

(72) Inventor: Jeffrey Gerard June, Oxford, OH (US)

(73) Assignee: Ischemia Care LLC, Oxford, OH (US)

(21) Appl. No.: 18/247,840

(22) PCT Filed: Oct. 4, 2021
(86) PCT No.: PCT/US2021/053300

§ 371 Date: Apr. 4, 2023

Related U.S. Application Data
(60) Provisional application No. 63/089,297, filed on Oct. 8, 2020.

(51) Int. Cl. C12Q 1/6883; G01N 33/68

(52) U.S. Cl. CPC C12Q 1/6883; G01N 33/6893; C12Q 2600/158; G01N 2800/2871

(57) ABSTRACT

The present invention discloses a method for diagnosing stroke utilizing gene expression signatures. The method facilitates to distinguish a body fluid sample of a subject suffered from stroke and a body fluid of a control sample from a subject not likely to suffer from stroke. The method further enables to differentiate cardioembolic stroke from large artery atherosclerosis stroke in the subject. The method further enables to detect a presence of atrial fibrillation in the subject. The method further enables to detect the presence of atrial fibrillation from cardioembolic stroke, and not due to atrial fibrillation and large artery atherosclerosis stroke.

### CLAIM OF PRIORITY UNDER 35 U.S.C. § 119

The present Application for Patent claims priority to U.S. Provisional Application No. 63/089,297 entitled “METHOD FOR DIAGNOSING STROKE UTILIZING GENE EXPRESSION SIGNATURES” filed Oct. 8, 2020, and hereby expressly incorporated by reference for all purposes herein.

### TECHNICAL FIELD

The present disclosure relates generally to the fields of cardiovascular disease, ischemic stroke, and biomarker detection. More specifically, the invention discloses biomarkers that are present in peripheral blood which are indicative of an increased risk for stroke and methods of use thereof in diagnostic and prognostic assays.

### BACKGROUND

Stroke has the third highest death-rate in industrial countries. In United States, annually, there are 795,000 stroke cases, of which 690,000 are ischemic stroke cases. Further, there are over 2 million emergency department hospital visits for stroke and “stroke like” symptoms. 185,000 ischemic stroke cases are recurrent events that are often more massive, debilitating, and severe. In 10% to 40% of ischemic stroke cases, the cause of stroke is never known, diagnosed as “cryptogenic”, leading to increased costs (both during hospitalization and post), higher recurrence risk, increased payor costs, clinical frustration, and poor patient satisfaction and anxiety. Annually, 34 billion dollars are spent on diagnosis and treatment of stroke.

Stroke is caused either by bleeding in the brain from a ruptured blood vessel such as hemorrhagic stroke or by obstruction of a blood vessel in the brain which could be ischemic or thrombotic stroke. Ischemic stroke results from either a permanent or a transient reduction in cerebral blood flow. This reduction in flow is, in most cases, caused by the arterial occlusion due to either an embolus or a local thrombosis. Depending on the localization of brain injury and the intensity of necrosed neurons, stroke symptoms can become a life handicap for patients.

Strokes can be categorized based on their location and nature. However, some strokes, such as intracranial hemorrhages, are unpredictable. Others, such as small deep strokes, are frequently too minor to notice. However, a significant portion of strokes is large, surface-based, and ischemic, meaning they are caused by a blockage in a large artery. Such a potentially debilitating stroke has the potential to be identified at the time of occurrence or shortly thereafter. This potential for rapid detection is medically critical since the time elapsed from stroke onset until medical treatment is the most significant variable factor affecting the successful treatment of an acute stroke. In short, medical treatment administered quickly is critical for either preventing or significantly ameliorating the medical impact of a stroke on a patient.

Clearly, a need exists in the art for a panel of biomarkers associated with an increased risk of stroke for the management and treatment thereof.

Therefore, there is a need for a method and blood testing panels in the acute setting to determine (i) if a stroke has occurred, (ii) differentiation between cardioembolic stroke (CES) and large artery atherosclerosis stroke (LAA), and (iii) detect the presence of atrial fibrillation (AF).

### BRIEF SUMMARY OF THE INVENTION

The present invention discloses a method for diagnosing stroke using gene expression signatures. The method facilitates differentiating a body fluid sample of a subject that suffered from stroke and a body fluid of a control sample from a subject not likely to have suffered from stroke (but is risk factor matched with the stroke population). The method further facilitates differentiating cardioembolic stroke (CES) from large artery atherosclerosis stroke (LAA) in the subject. The method further facilitates the detection of the presence of atrial fibrillation (AF) in the subject. If atrial fibrillation is detected, then the method further facilitates detection of whether the stroke from (a) cardioembolic stroke (CES) not due to atrial fibrillation or (b) large artery atherosclerosis stroke (LAA).

In one embodiment, the method for diagnosing stroke is disclosed. The method involves: distinguishing a body fluid sample of a subject that has suffered from stroke and a body fluid control sample from a subject not likely to have suffered from stroke; differentiating cardioembolic stroke from large artery atherosclerosis stroke in the subject; and detecting the presence of atrial fibrillation (AF) in the subject.

In accordance with the present invention, a method for identifying patients having an increased risk for the development of acute ischemic stroke is provided. An exemplary method entails obtaining a biological sample from a test subject and determining the expression levels of at least twelve gene markers from Table 1, wherein upregulation of the markers relative to predetermined control levels observed in non-afflicted controls, are indicative of an increased risk for the development of acute ischemic stroke. In one embodiment the three gene markers are MIR3926-1, CETN2, CAPRIN1, MIR3677, NPRL3, CHTF8, NACA2, EEF2, FPR1, OLIG1, APOBEC3B-AS1, and LINC00229. The method may further comprise analysis of FCGR2C, LOC729732, and CD63. In yet another embodiment the gene markers further comprise of FCGR2C, BANK1, LOC729732, DUSP16, CD63, FLVCR2, HIC1, and CRLS1.

In one or more embodiments, the predetermined levels are mean expression levels across the patient cohort. In one or more embodiments, the determining step comprises contacting the sample with an agent having affinity for the ischemic stroke associated markers, the agent forming a specific binding pair with the markers and further comprising a detectable label, measuring the detectable label, thereby determining expression level of the marker in the sample. In one or more embodiments, the expression levels are determined using an input quantity method. In one or more embodiments, the markers are selected from the group consisting of polypeptides, nucleic acids or fragments thereof. In one or more embodiments, the molecules comprise polypeptides or fragments thereof, the agent is an antibody or fragment thereof and the polypeptide is detected by a method selected from the group consisting of flow cytometric analysis, immunohisto-chemical detection and immunoblot analysis.

In one or more embodiments, the molecules comprise nucleic acids or fragments thereof, the agent is complementary nucleic acids which hybridizes to the molecules and the ischemic stroke associated nucleic acid is detected by a method selected from the group consisting of in situ hybridization assay, hybridization assay, gel electrophoresis, RT-PCR, real time PCR, and microarray analysis. In one or more embodiments, the biological sample is a peripheral blood sample. In one or more embodiments, the methods further comprise creating a report summarizing the data obtained by the determination of the ischemic stroke associated marker expression levels. In one or more embodiments, the report includes recommendation for a treatment modality of the patient. In one or more embodiments, the patient is diagnosed with acute ischemic stroke and is undergoing treatment for the stroke.

In one embodiment, at least twelve gene signatures for distinguishing stroke from control is provided. The symbol list includes MIR3926-1, CETN2, CAPRIN1, MIR3677, NPRL3, CHTF8, NACA2, EEF2, FPR1, OLIG1, APOBEC3B-AS1, and LINC00229, and the accession list includes NR_037492, NM_004344, NM_005898, NR_037448, NM_001039476, NR_033227, NM_199290, NM_001961, M37128, BC011252, ENST00000513758, and NR_044991.

In one embodiment, at least three gene signatures for distinguishing cardioembolic stroke (CES) from large artery atherosclerosis stroke (LAA) is provided. The symbol list includes FCGR2C, LOC729732, and CD63, and the accession list includes NM_201563, AK124896, and NM_001040034.

In one embodiment, at least eight gene signatures for distinguishing the presence of atrial fibrillation (AF) from (a) cardioembolic stroke (CES) not due to atrial fibrillation and (b) large artery atherosclerosis stroke (LAA) is provided. The symbol list includes FCGR2C, BANK1, LOC729732, DUSP16, CD63, FLVCR2, HIC1, and CRLS1. The accession list includes NM_201563, NM_001083907, AK124896, NM_030640, NM_001040034, NM_001195283, AJ550616, and NM_001127458.

In one or more embodiments, the methods of the present invention further comprise determination of expression levels of any of the markers listed in Table 1.

In certain aspects, the determining step comprises contacting the sample with an agent having affinity for the ischemic stroke associated markers, the agent forming a specific binding pair with the markers and further comprising a detectable label, measuring the detectable label, thereby determining expression level of the marker in the sample. In a particularly preferred embodiment, the expression levels are determined using the input quantity method described in below. The method may further comprises creating a report summarizing the data obtained by the determination of the ischemic stroke associated marker expression levels and may include recommendation for a treatment modality of the patient.

In yet another aspect of the invention, kits are provided for practicing the methods disclosed herein.

In one or more embodiments, the present invention provides for a method for identifying agents which useful for the treatment of acute ischemic stroke, comprising: a) contacting a cell comprising one or more ischemic stroke associated markers from Table 1 with a test agent; and b) assessing the effect of the agent on modulation of expression levels of the markers relative to untreated cells, agents which modulate expression of any of the markers in step a) having utility for the treatment of acute ischemic stroke. In one or more embodiments, the present invention provides for a method for assessing an increased risk for the development acute ischemic stroke in a human patient and administering treatment for ameliorating symptoms of the stroke if necessary to the patient, comprising: a) obtaining a peripheral blood sample from the patient; b) determining the expression levels of ischemic stroke associated markers disclosed here, wherein upregulation of the markers relative to predetermined control levels observed in stroke afflicted subjects are indicative of an increased risk for the development of acute ischemic stroke; and c) administering an agent useful for the amelioration of stroke symptoms to the patient.

The invention also provides a method for identifying agents which useful for the treatment of acute ischemic stroke. An exemplary method comprises contacting a cell comprising one or more ischemic stroke associated markers from Table 1 with a test agent and assessing the effect of the agent on modulation of expression levels of the markers relative to untreated cells, agents which modulate expression of any of the markers in step a) having utility for the treatment of acute ischemic stroke.

In another embodiment, the invention provides a “test and treat” method for acute ischemic stroke. The patient is first assessed for the expression levels of the ischemic stroke associated markers described above, and if the marker profile is indicative of the presence or predisposition towards a stroke, the patient is administered treatment and placed on the appropriate therapeutic regimen.

The above summary contains simplifications, generalizations and omissions of detail and is not intended as a comprehensive description of the claimed subject matter but, rather, is intended to provide a brief overview of some of the functionality associated therewith. Other systems, methods, functionality, features and advantages of the claimed subject matter will be or will become apparent to one with skill in the art upon examination of the following figures and detailed written description.

The description of the illustrative embodiments can be read in conjunction with the accompanying figures. It will be appreciated that for simplicity and clarity of illustration, elements illustrated in the figures have not necessarily been drawn to scale. For example, the dimensions of some of the elements are exaggerated relative to other elements. Embodiments incorporating teachings of the present disclosure are shown and described with respect to the figures presented herein, in which:

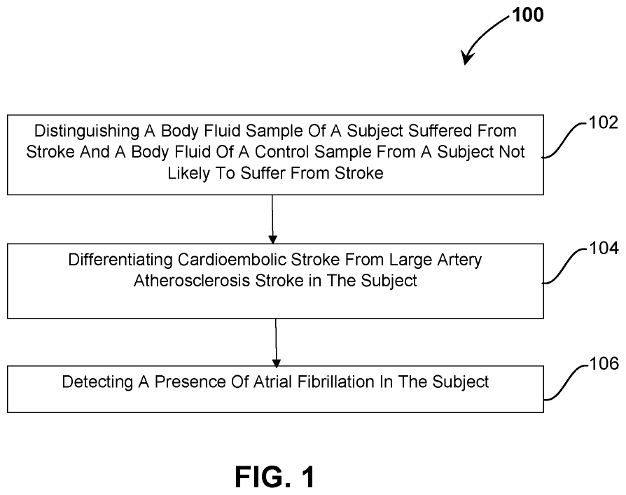

FIG. 1 exemplarily illustrates a flowchart of a method for diagnosing stroke utilizing gene expression signatures, according to an embodiment of the present invention.

### DETAILED DESCRIPTION OF EXAMPLE EMBODIMENTS

The terms “agent” and “test compound” are used interchangeably herein and denote a chemical compound, a mixture of chemical compounds, a biological macromolecule, or an extract made from biological materials such as bacteria, plants, fungi, or animal (particularly mammalian) cells or tissues. Biological macromolecules include siRNA, shRNA, antisense oligonucleotides, small molecules, antibodies, peptides, peptide/DNA complexes, and any nucleic acid-based molecule, for example an oligo, which exhibits the capacity to modulate the activity of the genetic signature nucleic acids described herein or their encoded proteins. Agents are evaluated for potential biological activity by inclusion in screening assays described herein below.

The biomarkers of the invention include genes and proteins, and variants and fragments thereof. Such biomarkers include DNA comprising the entire or partial sequence of the nucleic acid sequence encoding the biomarker, or the complement of such a sequence. The biomarker nucleic acids also include RNA comprising the entire or partial sequence of any of the nucleic acid sequences of interest. A biomarker protein is a protein encoded by or corresponding to a DNA biomarker of the invention. A biomarker protein comprises the entire or partial amino acid sequence of any of the biomarker proteins or polypeptides.

For purposes of the present invention, “a” or “an” entity refers to one or more of that entity; for example, “a cDNA” refers to one or more cDNA or at least one cDNA. The terms “a” or “an,” “one or more” and “at least one” can be used interchangeably herein. It is also noted that the terms “comprising,” “including,” and “having” can be used interchangeably. Furthermore, a compound “selected from the group consisting of” refers to one or more of the compounds in the list that follows, including mixtures (i.e. combinations) of two or more of the compounds. According to the present invention, an isolated, or biologically pure molecule is a compound that has been removed from its natural milieu. As such, “isolated” and “biologically pure@ do not necessarily reflect the extent to which the compound has been purified. An isolated compound of the present invention can be obtained from its natural source, can be produced using laboratory synthetic techniques or can be produced by any such chemical synthetic route. The term “genetic alteration” as used herein refers to a change from the wild-type or reference sequence of one or more nucleic acid molecules. Genetic alterations include without limitation, base pair substitutions, additions and deletions of at least one nucleotide from a nucleic acid molecule of known sequence.

A “biomarker” is any gene or protein whose level of expression in a tissue or cell is altered compared to that of a normal or healthy cell or tissue. Biomarkers of the invention are selective for underlying risk of progression to ischemic stroke. By “selectively overexpressed in peripheral blood” is intended that the biomarker of interest is overexpressed in peripheral blood in stroke patients relative to levels observed in control patients. Thus, detection of the biomarkers of the invention permits the differentiation of samples indicative of increased risk of ischemic stroke. Biomarker profiles for this purpose are also within the scope of the invention.

The phrase “consisting essentially of” when referring to a particular nucleotide or amino acid means a sequence having the properties of a given SEQ ID NO. For example, when used in reference to an amino acid sequence, the phrase includes the sequence per se and molecular modifications that would not affect the functional and novel characteristics of the sequence.

The term “complementary” describes two nucleotides that can form multiple favorable interactions with one another. For example, adenine is complementary to thymine as they can form two hydrogen bonds. Similarly, guanine and cytosine are complementary since they can form three hydrogen bonds. Thus, if a nucleic acid sequence contains the following sequence of bases, thymine, adenine, guanine and cytosine, a “complement” of this nucleic acid molecule would be a molecule containing adenine in the place of thymine, thymine in the place of adenine, cytosine in the place of guanine, and guanine in the place of cytosine. Because the complement can contain a nucleic acid sequence that forms optimal interactions with the parent nucleic acid molecule, such a complement can bind with high affinity to its parent molecule. With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence. For example, specific hybridization can refer to a sequence which hybridizes to any specific marker gene or nucleic acid, but does not hybridize to other human nucleotides. In addition, a polynucleotide which “specifically hybridizes” may hybridize only to a specific marker, such a genetic signature-specific marker shown below. Appropriate conditions enabling specific hybridization of single stranded nucleic acid molecules of varying complementarity are well known in the art.

By the use of the term “enriched” in reference to nucleic acid it is meant that the specific DNA or RNA sequence constitutes a significantly higher fraction (2-5-fold) of the total DNA or RNA present in the cells or solution of interest than in normal cells or in the cells from which the sequence was taken. This could be caused by a person by preferential reduction in the amount of other DNA or RNA present, or by a preferential increase in the amount of the specific DNA or RNA sequence, or by a combination of the two. However, it should be noted that “enriched” does not imply that there are no other DNA or RNA sequences present, just that the relative amount of the sequence of interest has been significantly increased. It is also advantageous for some purposes that a nucleotide sequence be in purified form. The term “purified” in reference to nucleic acid does not require absolute purity (such as a homogeneous preparation); instead, it represents an indication that the sequence is relatively purer than in the natural environment (compared to the natural level, this level should be at least 2 to 5-fold greater, e.g., in terms of mg/ml). Individual clones isolated from a cDNA library may be purified to electrophoretic homogeneity. The claimed DNA molecules obtained from these clones can be obtained directly from total DNA or from total RNA. The cDNA clones are not naturally occurring, but rather are preferably obtained via manipulation of a partially purified naturally occurring substance (messenger RNA). The construction of a cDNA library from mRNA involves the creation of a synthetic substance (cDNA) and pure individual cDNA clones can be isolated from the synthetic library by clonal selection of the cells carrying the cDNA library. Thus, the process which includes the construction of a cDNA library from mRNA and isolation of distinct cDNA clones yields an approximately 10.sup.-6-fold purification of the native message. Thus, purification of at least one order of magnitude, preferably two or three orders, and more preferably four or five orders of magnitude is expressly contemplated. Thus, the term “substantially pure” refers to a preparation comprising at least 50-60% by weight the compound of interest (e.g., nucleic acid, oligonucleotide, etc.). More preferably, the preparation comprises at least 75% by weight, and most preferably 90-99% by weight, the compound of interest. Purity is measured by methods appropriate for the compound of interest.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

The phrase “genetic signature” refers to a plurality of nucleic acid molecules whose expression levels are indicative of a given metabolic or pathological state. The genetic signatures described herein can be employed to characterize at the molecular level the biomarker profile that is associated with an increased risk of ischemic stroke, thus providing a useful molecular tool for predicting outcomes, for identifying patients at risk, and for use in biomarker in assays for evaluating ischemic stroke preventive agents.

With regard to nucleic acids used in the invention, the term “isolated nucleic acid” is sometimes employed. This term, when applied to DNA, refers to a DNA molecule that is separated from sequences with which it is immediately contiguous (in the 5′ and 3′ directions) in the naturally occurring genome of the organism from which it was derived. For example, the “isolated nucleic acid” may comprise a DNA molecule inserted into a vector, such as a plasmid or virus vector, or integrated into the genomic DNA of a prokaryote or eukaryote. An “isolated nucleic acid molecule” may also comprise a cDNA molecule. An isolated nucleic acid molecule inserted into a vector is also sometimes referred to herein as a recombinant nucleic acid molecule.

With respect to RNA molecules, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form.

The term “modulates” as used herein refers increasing or decreasing. For example, the term modulate refers to the ability of a compound or test agent to either interfere with, or augment signaling or activity of a gene or protein of the present invention.

The term “oligonucleotide” or “oligo” as used herein means a short sequence of DNA or DNA derivatives typically 8 to 35 nucleotides in length, primers, or probes. An oligonucleotide can be derived synthetically, by cloning or by amplification. An oligo is defined as a nucleic acid molecule comprised of two or more ribo- or deoxyribonucleotides, preferably more than three. The exact size of the oligonucleotide will depend on various factors and on the particular application and use of the oligonucleotide. The term “derivative” is intended to include any of the above-described variants when comprising an additional chemical moiety not normally a part of these molecules. These chemical moieties can have varying purposes including, improving solubility, absorption, biological half-life, decreasing toxicity and eliminating or decreasing undesirable side effects.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Polymerase chain reaction (PCR) has been described in U.S. Pat. Nos. 4,683,195, 4,800,195, and 4,965,188, the entire disclosures of which are incorporated by reference herein.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “promoter element” describes a nucleotide sequence that is incorporated into a vector that, once inside an appropriate cell, can facilitate transcription factor and/or polymerase binding and subsequent transcription of portions of the vector DNA into mRNA. In one embodiment, the promoter element of the present invention precedes the 5′ end of the Ischemic Stroke specific marker nucleic acid molecule(s) such that the latter is transcribed into mRNA. Host cell machinery then translates mRNA into a polypeptide.

A “replicon” is any genetic element, for example, a plasmid, cosmid, bacmid, plastid, phage or virus that is capable of replication largely under its own control. A replicon may be either RNA or DNA and may be single or double stranded.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by colorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

A “siRNA” refers to a molecule involved in the RNA interference process for a sequence-specific post-transcriptional gene silencing or gene knockdown by providing small interfering RNAs (siRNAs) that has homology with the sequence of the targeted gene. Small interfering RNAs (siRNAs) can be synthesized in vitro or generated by ribonuclease III cleavage from longer dsRNA and are the mediators of sequence-specific mRNA degradation. Preferably, the siRNA of the invention are chemically synthesized using appropriately protected ribonucleoside phosphoramidites and a conventional DNA/RNA synthesizer. The siRNA can be synthesized as two separate, complementary RNA molecules, or as a single RNA molecule with two complementary regions. Commercial suppliers of synthetic RNA molecules or synthesis reagents include Applied Biosystems (Foster City, Calif, USA), Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA) and Cruachem (Glasgow, UK). Specific siRNA constructs for inhibiting elevated mRNA levels associated with Ischemic Stroke may be between 15-35 nucleotides in length, and more typically about 21 nucleotides in length.

As used herein, the term “risk” refers to an aspect of personal behavior, or lifestyle, an environmental exposure, or an inborn or inherited characteristic which on the basis of epidemiological evidence is known to be associated with health-related condition(s) considered important to ameliorate or prevent.

“Sample” or “patient sample” or “biological sample” generally refers to a sample which may be tested for a particular molecule, preferably a genetic signature specific marker molecule, such as a marker shown in the tables provided below. Samples may include but are not limited to peripheral blood cells, CNS fluids, serum, plasma, buccal swabs, urine, saliva, tears, pleural fluid and the like.

The term “solid matrix” as used herein refers to any format, such as beads, microparticles, a microarray, the surface of a microtitration well or a test tube, a dipstick or a filter. The material of the matrix may be polystyrene, cellulose, latex, nitrocellulose, nylon, polyacrylamide, dextran or agarose.

Many techniques are available to those skilled in the art to facilitate transformation, transfection, or transduction of the expression construct into a prokaryotic or eukaryotic organism. The terms “transformation”, “transfection”, and “transduction” refer to methods of inserting a nucleic acid and/or expression construct into a cell or host organism. These methods involve a variety of techniques, such as treating the cells with high concentrations of salt, an electric field, or detergent, to render the host cell outer membrane or wall permeable to nucleic acid molecules of interest, microinjection, peptide-tethering, PEG-fusion, and the like.

The term “vector” relates to a single or double stranded circular nucleic acid molecule that can be infected, transfected or transformed into cells and replicate independently or within the host cell genome. A circular double stranded nucleic acid molecule can be cut and thereby linearized upon treatment with restriction enzymes. An assortment of vectors, restriction enzymes, and the knowledge of the nucleotide sequences that are targeted by restriction enzymes are readily available to those skilled in the art, and include any replicon, such as a plasmid, cosmid, bacmid, phage or virus, to which another genetic sequence or element (either DNA or RNA) may be attached so as to bring about the replication of the attached sequence or element. A nucleic acid molecule of the invention can be inserted into a vector by cutting the vector with restriction enzymes and ligating the two pieces together.

Those skilled in the art will recognize that a nucleic acid vector can contain nucleic acid elements other than the promoter element and the Ischemic Stroke specific marker gene nucleic acid molecule(s). These other nucleic acid elements include, but are not limited to, origins of replication, ribosomal binding sites, nucleic acid sequences encoding drug resistance enzymes or amino acid metabolic enzymes, and nucleic acid sequences encoding secretion signals, localization signals, or signals useful for polypeptide purification.

The present invention discloses a method for diagnosing stroke utilizing gene expression signatures. The present invention further discloses a method and blood testing panels in the acute setting to determine:
i. if a stroke has occurred;
ii. differentiation between cardioembolic stroke (CES) and large artery atherosclerosis stroke (LAA); and
iii. detect the presence of atrial fibrillation (AF).

In one embodiment, the method provides a diagnostic blood test to distinguish between a patient who has had a stroke from a patient who has not had a stroke. In another embodiment, the method provides a diagnostic blood test to differentiate between cardioembolic stroke (CES) or large artery atherosclerosis stroke (LAA). In yet another embodiment, the method provides a diagnostic blood test to separately detect the presence of atrial fibrillation (AF).

FIG. 1 exemplarily illustrates a flowchart of a method 100 for diagnosing stroke utilizing gene expression signatures, according to an embodiment of the present invention. At step 102, a body fluid sample of a subject that has suffered from stroke is distinguished from a body fluid control sample from a subject not likely to have suffered from stroke. At step 104, the method 100 involves differentiating a cardioembolic stroke from a large artery atherosclerosis stroke in the subject. At step 106, the method 100 involves detecting the presence of atrial fibrillation (AF) in the subject.

In another embodiment, the method involves drawing blood in the emergency department for further tests. The testing detects the changes in the blood and the type of stroke including a cardioembolic stroke and a large artery atherosclerosis stroke. The clinicians use results to stratify into downstream decisions. The method of the present invention reduces cost, improves care, leverages data and manages chronic condition. The method of the present invention facilitates identification of causes and prevention of stroke, improvement in clinical workflow decisions, impact spend downstream of testing, and provides gold standard accuracy.

In one embodiment, at least twelve gene signatures are used for distinguishing stroke from control are provided. In one embodiment, the gene signatures for distinguishing stroke comprises MIR3926-1, CETN2, CAPRIN1, MIR3677, NPRL3, CHTF8, NACA2, EEF2, FPR1, OLIG1, APOBEC3B-AS1, and LINC00229 (gene accession list NR_037492, NM_004344, NM_005898, NR_037448, NM_001039476, NR_033227, NM_199290, NM_001961, M37128, BC011252, ENST00000513758, and NR_044991).

In one or more embodiments, the methods of the present invention further comprise an additional test of at least three gene signatures for distinguishing a cardioembolic stroke (CES) from a large artery atherosclerosis stroke (LAA) is provided. The symbol list includes FCGR2C, LOC729732, and CD63, and the accession list includes NM_201563, AK124896, and NM_001040034.

In one embodiment, at least eight gene signatures for distinguishing the presence of atrial fibrillation (AF) from (a) cardioembolic stroke (CES) not due to atrial fibrillation and (b) large artery atherosclerosis stroke (LAA) is provided. The symbol list includes FCGR2C, BANK1, LOC729732, DUSP16, CD63, FLVCR2, HIC1, and CRLS1. The accession list includes NM_201563, NM_001083907, AK124896, NM_030640, NM_001040034, NM_001195283, AJ550616, and NM_001127458.

TABLE 1
Biomarkers for Distinguishing Stroke
Gene Signatures For Distinguishing Stroke

| MIR3926-1 | NR_037492 |
|---|---|
| CETN2 | NM_004344 |
| CAPRIN1 | NM_005898 |
| MIR3677 | NR_037448 |
| NPRL3 | NM_001039476 |
| CHTF8 | NR_033227 |
| NACA2 | NM_199290 |
| EEF2 | NM_001961 |
| FPR1 | M37128 |
| OLIG1 | BC011252 |
| APOBEC3B- | ENST00000513758 |
| AS1 | NR_044991). |
| LINC00229 | |
| Gene Signatures For Distinguishing A | |
| Cardioembolic Stroke (CES) From A | |
| Large Artery Atherosclerosis Stroke | |
| (LAA) | |
| FCGR2C | NM_201563 |
| LOC729732 | AK124896 |
| CD63 | NM_001040034 |
| Gene Signatures For Distinguishing The | |
| Presence Of Atrial Fibrillation (AF) From | |
| (A) Cardioembolic Stroke (CES) Not Due | |
| To Atrial Fibrillation And (B) Large | |
| Artery Atherosclerosis Stroke (LAA) | |
| FCGR2C | NM_201563 |
| BANK1 | NM_001083907 |
| LOC729732 | AK124896 |
| DUSP16 | NM_030640 |
| CD63 | NM_001040034 |
| FLVCR2 | NM_001195283 |
| HIC1 | AJ550616 |
| CRLS1 | NM_001127458 |

### Example 1

In another embodiment, the method for diagnosing the stroke is disclosed as follows. Initially, the biomarkers of acute stroke etiology (BASE) trial enrolled suspected stroke patients presenting to 20 hospitals within 24 hours of symptom onset. Age, race, gender, smoking, comorbidity match controls were all recoded. Final gold standard diagnosis and stroke etiology were determined by an adjudication committee using all hospital data but blinded to RNA test results. The whole blood, obtained in PAX tubes, was frozen at −20C within 72 hours and analyzed at a core lab (Ischemia Care, Dayton, OH) using Affymetrix Human Transcriptome microarrays (HTA). Genes on the HTA microarray were filtered to eliminate genes with low expression or high coefficients of gene expression variation (>10%) when run on replicate samples leaving 9,513 potential signature genes. A two-way random forest classifier was built through cross validation of the training data resulting in three distinct diagnostic signatures based upon 23 genes for a panel of three separate tests.

The results and conclusions of the method of diagnosis are discussed as follows. This was a planned interim cohort study of the 1700 patients enrolled in the BASE trial that does not include lacunar strokes, cryptogenic strokes, TIA, or stroke mimics. Overall, 224 patients were enrolled with NIHSS>=5, 59 (26%) with LAS, 165 (74%) with CES, and 66 control subjects; 56% were male, and median (IQR) age was 72.9 years (63.7, 82.9). Median (IQR) time from symptom onset to blood collection was 487 (321, 1129) minutes. Coexistent pathology at presentation included atrial fibrillation 120 (54%), hypertension 186 (83%), hyperlipidemia 186 (48%), diabetes 74 (33%), and coronary artery disease 78 (35%). Patients were randomly divided into training (132) and validation (92). The diagnostic 12 gene signature results distinguished stroke from control; C-statistic 0.86, sensitivity of 0.91, specificity of 0.61. The diagnostic 3 gene signature results distinguished CES from LAA C-statistic 0.70, sensitivity of 0.85, specificity of 0.49. The diagnostic 8 gene signature results distinguished AF from (a) cardioembolic stroke not due to atrial fibrillation and (b) LAA, C-statistic 0.69, sensitivity of 0.70, specificity of 0.59.

Thus, targeted panels of RNA expression markers may be used together or separately to determine if a stroke occurred, stratification into CE and LAA causes, and presence of AF, and may have triage, interventional, therapeutic, and secondary prevention implications.

Genetic signature or biomarker encoding nucleic acids, including but not limited to those listed may be used for a variety of purposes in accordance with the present invention. The genetic signature associated with an increased risk of ischemic stroke (e.g., the plurality of nucleic acids contained therein) containing DNA, RNA, or fragments thereof may be used as probes to detect the presence of and/or expression of these specific markers in a biological sample. Methods in which such marker nucleic acids may be utilized as probes for such assays include, but are not limited to: (1) in situ hybridization; (2) Southern hybridization (3) northern hybridization; and (4) assorted amplification reactions such as high throughput reverse transcription, quantitative polymerase chain reactions (HT RT qPCR) or conventional PCR.

Further, assays for detecting the genetic signature may be conducted on any type of biological sample, but is most preferably performed on peripheral blood. From the foregoing discussion, it can be seen that genetic signature containing nucleic acids, vectors expressing the same, genetic signature encoded proteins and anti-genetic signature encoded protein specific antibodies of the invention can be used to detect the signature in body tissue, cells, or fluid, and alter genetic signature containing marker protein expression for purposes of assessing the genetic and protein interactions involved in ischemic stroke.

In certain embodiments for screening for genetic signature containing nucleic acid(s), the sample will initially be amplified, e.g. using high throughput RT-qPCR, to increase the amount of the template as compared to other sequences present in the sample. This allows the target sequences to be detected with a high degree of sensitivity if they are present in the sample. This initial step may be avoided by using highly sensitive array techniques that are becoming increasingly important in the art.

Alternatively, additional detection technologies can be employed which detect the ischemic stroke biomarker proteins directly. Such methods include geLC/MS/MS proteomics analysis. This approach provides a full panel of the protein biomarkers present in the sample and allows the clinician to predict outcomes based on the panel of biomarkers present in a sample.

Thus, any of the aforementioned techniques may be used to detect or quantify genetic signature expression and or protein expression levels and accordingly, diagnose patient susceptibility for developing ischemic stroke.

Any of the aforementioned products can be incorporated into a kit which may contain genetic signature polynucleotides or one or more such markers immobilized on a DNA microarray, an oligonucleotide, a polypeptide, a peptide, an antibody, a label, marker, or reporter, a pharmaceutically acceptable carrier, a physiologically acceptable carrier, instructions for use, a container, a vessel for administration, an assay substrate, reagents and vessels suitable for obtaining a peripheral blood sample, reagents suitable for HT RT-qPCR, conventional PCR or any combination thereof. A DNA microarray (also commonly known as DNA chip or biochip) is a collection of microscopic DNA spots attached to a solid surface. Examples of providers for such microarrays includes Agilent with their Dual-Mode platform, Eppendorf with their DualChip platform for colorimetric Silverquant labeling, and TeleChem International with Arrayit. Several popular single-channel systems are the Affymetrix “Gene Chip”, Illumina “Bead Chip”, Agilent single-channel arrays, the Applied Microarrays “CodeLink” arrays, and the Eppendorf “DualChip & Silverquant”.

Since the genetic signature identified herein and the proteins encoded thereby have been associated with the etiology of ischemic stroke, methods for identifying agents that modulate the activity of the genes and their encoded products should result in the generation of efficacious therapeutic agents for the treatment of neurological and cardiovascular disorders, particularly those associated with ischemic stroke.

The nucleic acids comprising the signature contain regions which provide suitable targets for the rational design of therapeutic agents which modulate their activity. Small peptide molecules corresponding to these regions may be used to advantage in the design of therapeutic agents which effectively modulate the activity of the encoded proteins. Molecular modeling should facilitate the identification of specific organic molecules with capacity to bind to the active site of the proteins encoded by the genetic signature nucleic acids based on conformation or key amino acid residues required for function. A combinatorial chemistry approach will be used to identify molecules with greatest activity and then iterations of these molecules will be developed for further cycles of screening. In certain embodiments, candidate agents can be screening from large libraries of synthetic or natural compounds. Such compound libraries are commercially available from a number of companies including but not limited to Maybridge Chemical Co., (Trevillet, Cornwall, UK), Comgenex (Princeton, N.J.), Microsour (New Milford, Conn.) Aldrich (Milwaukee, Wis.) Akos Consulting and Solutions GmbH (Basel, Switzerland), Ambinter (Paris, France), Asinex (Moscow, Russia) Aurora (Graz, Austria), BioFocus DPI (Switzerland), Bionet (Camelford, UK), Chembridge (San Diego, Calif.), Chem Div (San Diego, Calif.). The skilled person is aware of other sources and can readily purchase the same. Once therapeutically efficacious compounds are identified in the screening assays described herein, they can be formulated into pharmaceutical compositions and utilized for the treatment of ischemic stroke patients.

The polypeptides or fragments employed in drug screening assays may either be free in solution, affixed to a solid support or within a cell. One method of drug screening utilizes eukaryotic or prokaryotic host cells which are stably transformed with recombinant polynucleotides expressing the biomarker polypeptide or fragment, preferably in competitive binding assays. Such cells, either in viable or fixed form, can be used for standard binding assays. One may determine, for example, formation of complexes between the polypeptide or fragment and the agent being tested, or examine the degree to which the formation of a complex between the polypeptide or fragment and a known substrate is interfered with by the agent being tested.

Another technique for drug screening provides high throughput screening for compounds having suitable binding affinity for the encoded polypeptides and is described in detail in Geysen, PCT published application WO 84/03564, published on Sep. 13, 1984. Briefly stated, large numbers of different, small peptide test compounds, such as those described above, are synthesized on a solid substrate, such as plastic pins or some other surface. The peptide test compounds are reacted with the target polypeptide and washed. Bound polypeptide is then detected by methods well known in the art.

A further technique for drug screening involves the use of host eukaryotic cell lines or cells (such as described above) which have a nonfunctional or altered ischemic stroke associated gene. These host cell lines or cells are defective at the polypeptide level. The host cell lines or cells are grown in the presence of drug compound. The effect on cell morphology and/or proliferation of the host cells is measured to determine if the compound is capable of regulating the same in the defective cells. Host cells contemplated for use in the present invention include but are not limited to bacterial cells, fungal cells, insect cells, mammalian cells, particularly neuronal, vascular, neutrophils, fibroblast, and CNS cells. The genetic signature encoding DNA molecules may be introduced singly into such host cells or in combination to assess the phenotype of cells conferred by such expression. Methods for introducing DNA molecules are also well known to those of ordinary skill in the art. Such methods are set forth in Ausubel et al. eds., Current Protocols in Molecular Biology, John Wiley & Sons, NY, N.Y. 1995, the disclosure of which is incorporated by reference herein.

Cells and cell lines suitable for studying the effects of genetic signature expression on cellular morphology and signaling methods of use thereof for drug discovery are provided. Such cells and cell lines will be transfected with one, two, three or all of the genetic signature encoding nucleic acids described herein and the effects on cell functions and cell signaling can be determined. Such cells and cell lines can also be contacted with the siRNA molecules provided herein to assess the effects thereof on similar functions. The siRNA molecules will be tested alone and in combination of 2, 3, 4, and 5 siRNAs to identify the most efficacious combination for down regulating target nucleic acids.

A wide variety of expression vectors are available that can be modified to express the novel DNA or RNA sequences of this invention. The specific vectors exemplified herein are merely illustrative, and are not intended to limit the scope of the invention. Expression methods are described by Sambrook et al. Molecular Cloning: A Laboratory Manual or Current Protocols in Molecular Biology 16.3-17.44 (1989). Expression methods in Saccharomyces are also described in Current Protocols in Molecular Biology (1989).

Suitable vectors for use in practicing the invention include prokaryotic vectors such as the pNH vectors (Stratagene Inc., 11099 N. Torrey Pines Rd., La Jolla, Calif. 92037), pET vectors (Novogen Inc., 565 Science Dr., Madison, Wis. 53711) and the pGEX vectors (Pharmacia LKB Biotechnology Inc., Piscataway, N.J. 08854). Examples of eukaryotic vectors useful in practicing the present invention include the vectors pRc/CMV, pRc/RSV, and pREP (Invitrogen, 11588 Sorrento Valley Rd., San Diego, Calif 92121); pcDNA3.1/V5&His (Invitrogen); baculovirus vectors such as pVL1392, pVL1393, or pAC360 (Invitrogen); and yeast vectors such as YRP17, YIPS, and YEP24 (New England Biolabs, Beverly, Mass.), as well as pRS403 and pRS413 Stratagene Inc.); Picchia vectors such as pHIL-D1 (Phillips Petroleum Co., Bartlesville, Okla. 74004); retroviral vectors such as PLNCX and pLPCX (Clontech); and adenoviral and adeno-associated viral vectors.

Promoters for use in expression vectors of this invention include promoters that are operable in prokaryotic or eukaryotic cells. Promoters that are operable in prokaryotic cells include lactose (lac) control elements, bacteriophage lambda (pL) control elements, arabinose control elements, tryptophan (trp) control elements, bacteriophage T7 control elements, and hybrids thereof. Promoters that are operable in eukaryotic cells include Epstein Barr virus promoters, adenovirus promoters, SV40 promoters, Rous Sarcoma Virus promoters, cytomegalovirus (CMV) promoters, baculovirus promoters such as AcMNPV polyhedrin promoter, Picchia promoters such as the alcohol oxidase promoter, and Saccharomyces promoters such as the gal4 inducible promoter and the PGK constitutive promoter, as well as neuronal-specific platelet-derived growth factor promoter (PDGF).

In addition, a vector of this invention may contain any one of a number of various markers facilitating the selection of a transformed host cell. Such markers include genes associated with temperature sensitivity, drug resistance, or enzymes associated with phenotypic characteristics of the host organisms.

Host cells expressing the genetic signature of the present invention or functional fragments thereof provide a system in which to screen potential compounds or agents for the ability to modulate the development of acute ischemic stroke

Another approach entails the use of phage display libraries engineered to express fragment of the polypeptides encoded by the genetic signature containing nucleic acids on the phage surface. Such libraries are then contacted with a combinatorial chemical library under conditions wherein binding affinity between the expressed peptide and the components of the chemical library may be detected. U.S. Pat. Nos. 6,057,098 and 5,965,456 provide methods and apparatus for performing such assays.

The goal of rational drug design is to produce structural analogs of biologically active polypeptides of interest or of small molecules with which they interact (e.g., agonists, antagonists, inhibitors) in order to fashion drugs which are, for example, more active or stable forms of the polypeptide, or which, e.g., enhance or interfere with the function of a polypeptide in vivo. See, e.g., Hodgson, (1991) Bio/Technology 9:19-21. In one approach, discussed above, the three-dimensional structure of a protein of interest or, for example, of the protein-substrate complex, is solved by x-ray crystallography, by nuclear magnetic resonance, by computer modeling or most typically, by a combination of approaches. Less often, useful information regarding the structure of a polypeptide may be gained by modeling based on the structure of homologous proteins. An example of rational drug design is the development of HIV protease inhibitors (Erickson et al., (1990) Science 249:527-533). In addition, peptides may be analyzed by an alanine scan (Wells, (1991) Meth. Enzym. 202:390-411). In this technique, an amino acid residue is replaced by Ala, and its effect on the peptide's activity is determined. Each of the amino acid residues of the peptide is analyzed in this manner to determine the important regions of the peptide.

It is also possible to isolate a target-specific antibody, selected by a functional assay, and then to solve its crystal structure. In principle, this approach yields a pharmacophore upon which subsequent drug design can be based.

One can bypass protein crystallography altogether by generating anti-idiotypic antibodies (anti-ids) to a functional, pharmacologically active antibody. As a mirror image of a mirror image, the binding site of the anti-ids would be expected to be an analog of the original molecule. The anti-id could then be used to identify and isolate peptides from banks of chemically or biologically produced banks of peptides. Selected peptides would then act as the pharmacophore.

Thus, one may design drugs which have, e.g., improved polypeptide activity or stability or which act as inhibitors, agonists, antagonists, etc. of polypeptide activity. By virtue of the availability of the genetic signature containing nucleic acid sequences described herein, sufficient amounts of the encoded polypeptide may be made available to perform such analytical studies as x-ray crystallography. In addition, the knowledge of the protein sequence provided herein will guide those employing computer modeling techniques in place of, or in addition to x-ray crystallography.

In another embodiment, the availability of genetic signature containing nucleic acids enables the production of strains of laboratory mice carrying the signature(s) of the invention. Transgenic mice expressing the genetic signature of the invention provide a model system in which to examine the role of the protein(s) encoded by the signature containing nucleic acid in the development and progression towards ischemic stroke. Methods of introducing transgenes in laboratory mice are known to those of skill in the art. Three common methods include: (1) integration of retroviral vectors encoding the foreign gene of interest into an early embryo; (2) injection of DNA into the pronucleus of a newly fertilized egg; and (3) the incorporation of genetically manipulated embryonic stem cells into an early embryo. Production of the transgenic mice described above will facilitate the molecular elucidation of the role that a target protein plays in various cellular metabolic processes. Such mice provide an in vivo screening tool to study putative therapeutic drugs in a whole animal model and are encompassed by the present invention.

The elucidation of the role played by the gene products described herein in ischemic stroke occurrence facilitates the development of pharmaceutical compositions useful for the diagnosis, management, and treatment of ischemic stroke. These compositions may comprise, in addition to one of the above substances, a pharmaceutically acceptable excipient, carrier, buffer, stabilizer or other materials well known to those skilled in the art. Such materials should be non-toxic and should not interfere with the efficacy of the active ingredient.

Whether it is a polypeptide, antibody, peptide, nucleic acid molecule, small molecule or other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual.

The method of the present invention allows the medical practitioners to identify cause for secondary prevention, identify cause and risk factor of atrial fibrillation, identify individuals that have suffered from stroke, and identify transient ischemic attacks and transient neurological events. The present invention increases efficiency across ambulatory, emergency room, neurology, cardiology, laboratory, payment, and outpatient care. Further, the present invention enables caregivers to focus on care decisions that leverage data, reduce costs, and manage chronic conditions.

While the disclosure has been described with reference to exemplary embodiments, it will be understood by those skilled in the art that various changes may be made and equivalents may be substituted for elements thereof without departing from the scope of the disclosure. In addition, many modifications may be made to adapt a particular system, device or component thereof to the teachings of the disclosure without departing from the essential scope thereof. Therefore, it is intended that the disclosure is not limited to the particular embodiments disclosed for carrying out this disclosure, but that the disclosure will include all embodiments falling within the scope of the appended claims. Moreover, the use of the terms first, second, etc. do not denote any order or importance, but rather the terms first, second, etc. are used to distinguish one element from another.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The description of the present disclosure has been presented for purposes of illustration and description, but is not intended to be exhaustive or limited to the disclosure in the form disclosed. Many modifications and variations will be apparent to those of ordinary skill in the art without departing from the scope of the disclosure. The described embodiments were chosen and described in order to best explain the principles of the disclosure and the practical application, and to enable others of ordinary skill in the art to understand the disclosure for various embodiments with various modifications as are suited to the particular use contemplated.

What is claimed is:

1. A method for diagnosing stroke using gene expression signatures, comprising the steps of:
a. distinguishing a body fluid sample of a subject that has suffered from stroke and a body fluid control sample from a subject not likely to have suffered from stroke;
b. differentiating cardioembolic stroke from large artery atherosclerosis stroke in the subject; and
c. detecting a presence of atrial fibrillation in the subject.

2. The method of claim 1, further comprises a step of detecting a presence of atrial fibrillation caused from cardioembolic stroke.

3. The method of claim 1, further comprises a step of detecting a presence of atrial fibrillation from cardioembolic stroke not due to atrial fibrillation and large artery atherosclerosis stroke.

4. The method of claim 1, wherein the body fluid sample is selected from at least one of blood, plasma, or serum.

5. The method of claim 1, wherein the gene expression signatures for distinguishing stroke subject from control sample are selected from a group comprising MIR3926-1, CETN2, CAPRIN1, MIR3677, NPRL3, CHTF8, NACA2, EEF2, FPR1, OLIG1, APOBEC3B-AS1, and LINC00229.

6. The method of claim 1, wherein the gene expression signatures for distinguishing cardioembolic stroke from large artery atherosclerosis stroke are selected from a group comprising FCGR2C, LOC729732, and CD63.

7. The method of claim 1, wherein the gene expression signatures for detecting the presence of atrial fibrillation are selected from the group comprising FCGR2C, BANK1, LOC729732, DUSP16, CD63, FLVCR2, HIC1 and CRLS1.

8. The method of claim 1, wherein the gene expression signatures for distinguishing the presence of atrial fibrillation from cardioembolic stroke not due to atrial fibrillation and large artery atherosclerosis stroke, are selected from the group comprising FCGR2C, BANK1, LOC729732, DUSP16, CD63, FLVCR2, HIC1 and CRLS1.

9. The method of claim 1, forms a two-way random forest classifier through cross validation of the training data resulting in three distinct diagnostic signatures based upon a plurality of genes for a panel of three separate tests.

10. The method of claim 1, utilizes targeted panels of RNA expression markers together or separately to determine occurrence of stroke, stratification into cardioembolic stroke and large artery atherosclerosis stroke causes, and presence of atrial fibrillation.
